(19) United States
(12) Patent Application Publication
(10) Pub. No.: US 2007/0184008 A1
(43) Pub. Date: Aug. 9, 2007

(54) Shea butter polyoxyalkylene glycol esters

(76) Inventors: Steven Rogers, Yardley, PA (US); Anthony O'Lenick JR., Dacula, GA (US)

Correspondence Address: Louis C. Paul & Associates, PLLC, 9th floor, 730 Fifth Avenue, New York, NY, 10019, US

(21) Appl. No.: 11/347,270

(22) Filed: Feb. 6, 2006

(51) Int. Cl. A61K 8/37

(52) U.S. Cl. 424/70.31; 554/227

(57) ABSTRACT

Novel polyoxyalkylene glycol esters prepared by the reaction of a polyoxyethylene glycol (PEG) or polyoxypropylene glycol (PPG) or mixtures thereof with shea butter, preferably mild-processed shea butter (MPSB) under specific mild-processing conditions. These materials are useful as cosmetic and personal care ingredients, allowing for the delivery of highly desirable active ingredients present in shea butter, including natural antioxidants, in a water-soluble form that is substantive to the skin and hair.

### CROSS-REFERENCE TO RELATED APPLICATIONS

Not applicable.

### Statement Regarding Federally-Sponsored Research or Development

Not applicable.

### Field of the Invention

The present invention relates to novel shea butter polyoxyalkylene glycol esters prepared by the reaction of a polyoxyethylene glycol (PEG) or polyoxypropylene glycol (PPG) or mixtures thereof with shea butter, preferably mild-processed shea butter (MPSB) under specific mild-processing conditions. These compounds are useful as cosmetic and personal care ingredients, allowing for the delivery of highly desirable active ingredients present in shea butter including natural antioxidants, in a water-soluble form that is substantive to the skin and hair.

### BACKGROUND OF THE INVENTION

Shea Butter is a butter extracted from the kernel of Butrospermum parkii. This plant, also referred to as Vitellaria paradoxa, is native to Africa. The term butter describes a material that is a solid at room temperature, but melts at about 40° C. Chemically, the butter is a triglyceride conforming to the following structure

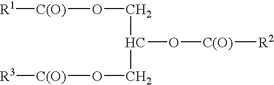

wherein R₁, R² and R³ each have one of the following compositions:

| R Group | Common Name | Range (%) | Typical (%) |
|---|---|---|---|
| C₁₁H₂₃ | Lauryl | 0.1-2.0 | 0.2 |
| C₁₃H₂₇ | Myristyl | 0.5-2.0 | 1.0 |
| C₁₅H₃₁ | Cetyl | 2.0-6.0 | 4.0 |
| C₁₇H₃₅ | Stearyl | 25.0-50.0 | 35.0 |
| C₁₇H₃₃ | Oleyl | 40.0-60.0 | 59.0 |
| C₁₇H₃₁ | Linoleyl | 0.5-1.0 | 0.8 |

The average composition of R² is different than R¹ and R³, the latter two being similar. The R² moiety contains predominantly the unsaturated C₁₈ group (oleyl) while R¹ and R³ contain predominantly the saturated C₁₈ group (stearyl). Differences between internal (R²) and terminal (R¹, R³) substitution are seen in natural products but not in synthetic molecules produced in the laboratory.

The high levels of stearyl and oleyl groups shea butter, particularly in mild-processed shea butter, and their polyoxyalkylene ester derivatives make them of particular interest in the personal care industry. While other raw materials used in personal care products have these species, the compounds of the present invention have significantly high concentrations of unsaponifiables, which posses highly desired antioxidant, ultra-violet radiation protection, and free-radical scavenging properties. MPSB of the present invention typically contains from about 5% to about 15% by weight of unsaponifiables. In contrast, other butters commonly used in personal care products have less than 2% unsaponifiables. For example, coca butter (from Theobroma cacao) averages 0.4% unsaponifiables and Illipe butter (from Shorea stenoptera) averages 1.1%.

As described in greater detail below, the novel polyoxyalkylene ester compounds of the present invention are produced by reacting shea butter (preferably MPSB under specific mild conditions) with polyoxyethylene glycol (PEG) or polyoxypropylene glycol (PPG) or mixtures thereof. By “mild processed” is meant processes that do not remove or otherwise diminish the amount or potency of active ingredients, particularly highly desired unsaponifiables. In the present invention, mild processing is employed both at the time of harvesting and initial extraction and during subsequent preparation of derivatives. These mild processes result in materials containing unexpectedly high amounts unsaponifiables, notably antioxidants.

As a class of surfactants, polyoxyalkylene glycol esters are known in the art. Monoesters as well as saturated, unsaturated and Guerbet diesters are commercially available. Differing hydrophile-lipophile balances (HLB) and melting points can be achieved by varying one or both of the length of the carbon chain in the fatty source and the molecular weight of the poloxyakylene group. For example, the HLB of lauric acid (C₁₂) diesters of PEG can vary from about 6 to about 18, where the molecular weights of the poloxyethylene glycol (the hydrophilic portion of the surfactant) ranges from PEG-200 to PEG-4000. PEG esters perform several functions, providing emulsification, acting as emollients and solubilizing fragrance oils.

U.S. Pat. No. 5,917,070 teaches polyoxyalkylene glycol esters prepared by the reaction of polyoxyalkylene glycol with Meadowfoam oil as a triglyceride, as a fatty acid or as a methyl ester. The compounds are said to have good oxidative stability related to the specific carbon chain distribution of Meadowfoam oil. The polyoxyalkylene glycol esters described in the '070 Patent do not possess the desirable unsaponifiable fractions, and with them antioxidant properties, of the compounds of the present invention.

### SUMMARY OF THE INVENTION

The compounds of the present invention are shea butter esters produced by reacting mild-process shea butter with polyoxyalkylene glycols. Preferably the shea butter is mild-processed and the reaction with the polyoxyalkylene glycols is under mild conditions. The novel compounds of the present invention are rich in unsaponifiables, including antioxidants.

### DETAILED DESCRIPTION OF THE INVENTION

The present invention relates to a novel class of polyoxyalkylene esters—namely PEG esters, PPG esters and PEG/PPG esters—made by reacting shea butter with polyoxyethylene glycol or polyoxypropylene glycol or mixtures thereof and a process for using them in personal care applications. Preferably, mild processing is employed both at the time of harvesting and initial extraction and during subsequent preparation of alkoxylate derivatives. In so doing, materials containing unexpectedly high amounts of active ingredients, particularly highly desired unsaponifiables, are produced.

Shea butter polyoxyalkylene glycol esters of the present invention conform to the following structure:
R—C(O)—(OCH₂CH₂)ₐ—(CH₂CH(CH₃)O)_{b}—(CH₂CH₂O)_{c}—(O)C—R

wherein
(i) R is derived from shea butter and comprises
   from about 0.1 to about 2.0% by weight C₁₁H₂₃;
   from about 0.5 to about 2.0% by weight C₁₃H₂₇;
   from about 2.0 to about 6.0% by weight C₁₅H₃₁;
   from about 25 to about 50% by weight C₁₇H₃₅;
   from about 40.0 to about 60.0% by weight C₁₇H₃₃; and
(ii) a, b and c are independently integers ranging from 1 to 20.

Another aspect of the invention is a process for delivering antioxidants to the skin and hair by applying thereto a finished topical product comprising an effective concentration of a shea butter polyoxyalkylene ester that is made by an esterification reaction and conforms to the structure:
R—C(O)—(OCH₂CH₂)ₐ—(CH₂CH(CH₃)O)_{b}—(CH₂CH₂O)_{c}—(O)C—R

wherein
(i) R is derived from shea butter and comprises
   from about 0.1 to about 2.0% by weight C₁₁H₂₃;
   from about 0.5 to about 2.0% by weight C₁₃H₂₇;
   from about 2.0 to about 6.0% by weight C₁₅H₃₁;
   from about 25 to about 50% by weight C₁₇H₃₅;
   from about 40.0 to about 60.0% by weight C₁₇H₃₃; and
(ii) a, b and c are independently integers ranging from 1 to 20.

In a preferred embodiment the esterification is conducted at a temperature of from about 180° C. to about 190° C. In another preferred embodiment, the effective concentration of MPSB polyoxyalkylene glycol ester is from about 0.5% to about 15.0% by weight of the total weight of the finished formulation.

Shea Butter

Shea butter can be prepared by standard extraction techniques known to those of skill in the art. For example, U.S. Pat. No. 6,552,208, the disclosure of which is incorporated herein by reference, describes several methods for processing shea butter. Suitable extraction vehicles may include, but are not limited to, ethanol, methanol, ethyl acetate, acetone, chloroform and water, or any other solvent and water.

In a preferred aspect of the present invention, shea butter is mild-processed; it is extracted using a hydrocarbon-free solvent system and its polyoxyalkylene ester derivatives of the present invention are made under mild processing conditions. At the time of harvesting and initial extraction ground-up kernels are boiled in water under mild conditions as described in the example below. The oil phase is then separated from the water phase by decanting. This process provides a yellow, solid wax rich in unsaponifiables. By wax is meant a material obtained by boiling in water under ambient conditions, decanted and filtered.

The mild processing of the present invention may be contrasted with separation using solvents and high temperature treatment with high pressure steam. While the latter processes result in what some may describe as a “more pure” triglyceride, unsaponifiables, and the benefits derived therefrom, are lost. Vacuum distillation which strips off the desirable components is also to be avoided in processing MPSB of the present invention. By processing shea butter under mild conditions, materials comprising from about 5% to about 15% by weight of unsaponifiables can be produced.

Sterols comprise about 20% of the unsaponifiables in shea butter. More particularly, the sterols comprise: cholesterol (from about 1% to about 3%); alpha-spinasterol (from about 1% to about 4%); delta-7-stigmasterol (from about 40% to about 44%); delta-7-avenasterol (from about 38% to about 41%). The remaining constituents of the unsaponifiables (about 80%) include other highly desirable active compounds including tocopherol, karitin, cinamic acid esters, alpha and beta amyrin and phenolics.

Phenolic compounds are natural products composed of one or more aromatic benzene rings with one or more hydroxyl group. They are a class of natural products that posses antioxidant and free radical scavenging properties. Among the phenolics in the unsaponifiables of mild-processed shea butter include gallic acid, gallocatchin, catechin, epigallocatechin gallate, epicatechin, gallocatechin gallate, gallocatechin gallate and quercetin.

Poloxyalkylene Glycols

Polyoxyalkylene glycols are an item of commerce and conform to the following structure:
H—(OCH₂CH₂)ₐ—(CH₂CH(CH₃)O)_{b}—(CH₂CH₂O)_{c}—H

wherein, a, b and c are independently integers ranging from 1 to 20. Illustrative, but not limiting, examples of loxalkylene glycols which may in preparing the esters of the present invention are set out below:

| Example | a | b | C |
|---|---|---|---|
| 1 | 1 | 0 | 0 |
| 2 | 10 | 0 | 0 |
| 3 | 0 | 10 | 0 |
| 4 | 5 | 1 | 5 |
| 5 | 5 | 10 | 10 |
| 6 | 20 | 20 | 20 |

The following examples are further illustrative of the present invention. The components and specific ingredients are presented as being typical, and various modifications can be derived in view of the foregoing disclosure within the scope of the invention. All percentages, ratios and proportions herein are by weight, unless otherwise specified. All temperatures are in degrees Celsius unless otherwise specified.

### EXAMPLES

The starting MPSB is made according to following procedure: 500.0 grams of the nut form the Shea Butter tree are cracked into small pieces and placed into a one-liter vat of water. The water is then heated to 100° C. As the temperature increases, an oil phase develops on the surface of the water. The temperature is held for about 2 hours, after which the oil is decanted and passed through filter paper. The resulting butter is mild-processed shea butter according to the present invention. It is rich in unsaponifiable (from about 70% to about 15% by weight) and may be used in making the MPSB polyoxyalklene glycol esters of the present invention.

Polyoxyalkylene glycols as described in Examples 1-6 are reacted with MPSB as follows: To 145.0 grams of the mild process shea butter are added the number of grams of polyoxyalkylene glycols in Examples 7-18. Using a reflux condenser, the temperature of the mass is raised to 180° C. -190° C. The mass is held within this temperature range for 10 hours. Nothing is distilled off during this time, and the reaction mass becomes hazy. The reaction mass is cooled, glycerin separates off and is removed from the bottom. The products of Example 7-18 may be used in formulating finished cosmetic and personal care products without additional purification.

| | Polyoxyalkylene Glycol | |
|---|---|---|
| Example | Example | Grams |
| 7 | 1 | 22.0 |
| 8 | 2 | 220.0 |
| 9 | 3 | 295.0 |
| 10 | 4 | 249.5 |
| 11 | 5 | 595.0 |
| 12 | 6 | 1470.0 |
| 13 | 1 | 33.0 |
| 14 | 2 | 330.0 |
| 15 | 3 | 442.5 |
| 16 | 4 | 3742.5 |
| 17 | 5 | 897.5 |
| 18 | 6 | 2205.0 |

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A shea butter polyoxyalkylene glycol ester conforming to the structure:
R—C(O)—(OCH₂CH₂)ₐ—(CH₂CH(CH₃)O)_{b}—(CH₂CH₂O)_{c}—(O)C—R
wherein
(i) R is derived from shea butter and comprises;
from about 0.1 to about 2.0% by weight C₁₁H₂₃;
from about 0.5 to about 2.0% by weight C₁₃H₂₇;
from about 2.0 to about 6.0% by weight C₁₅H₃₁;
from about 25 to about 50% by weight C₁₇H₃₅;
from about 40.0 to about 60.0% by weight C₁₇H₃₃; and
(ii) a, b and c are independently integers ranging from 1 to 20.

2. A shea butter polyoxyalkylene glycol ester of claim 1 wherein a is 1, b is 0 and c is 0.

3. A shea butter polyoxyalkylene glycol ester of claim 1 wherein a is 10, b is 0 and c is 0.

4. A shea butter polyoxyalkylene glycol ester of claim 1 wherein a is 0, b is 10 and c is 0.

5. A shea butter polyoxyalkylene glycol ester of claim 1 wherein a is 5, b is 1 and c is 5.

6. A shea butter polyoxyalkylene glycol ester of claim 1 wherein a is 5, b is 10 and c is 10.

7. A shea butter polyoxyalkylene glycol ester of claim 1 wherein a is 20, b is 20 and c is 20.

8. A process for delivering antioxidants to the skin and hair by applying thereto a finished topical product comprising an effective concentration of a shea butter polyoxyalkylene glycol ester that is made by an esterification reaction and conforms to the structure:
R—C(O)—(OCH₂CH₂)ₐ—(CH₂CH(CH₃)O)_{b}—(CH₂CH₂O)_{c}—(O)C—R
wherein
(i) R is derived from shea butter and comprises;
from about 0.1 to about 2.0% by weight C₁₁H₂₃;
from about 0.5 to about 2.0% by weight C₁₃H₂₇;
from about 2.0 to about 6.0% by weight C₁₅H ₃₁;
from about 25 to about 50% by weight C₁₇H₃₅;
from about 40.0 to about 60.0% by weight C₁₇H₃₃; and
(ii) a, b and c are independently integers ranging from 1 to 20.

9. A process of claim 8 wherein a is 1, b is 0 and c is 0.

10. A process of claim 8 wherein a is 10, b is 0 and c is 0.

11. A process of claim 8 wherein a is 0, b is 10 and c is 0.

12. A process of claim 8 wherein a is 5, b is 1 and c is 5.

13. A process of claim 8 wherein a is 5, b is 10 and c is 10.

14. A process of claim 8 wherein a is 20, b is 20 and c is 20.

15. A shea butter polyoxyalkylene glycol ester of claim 1 where the shea butter is mild-processed.

16. A process of claim 8 where the shea butter is mild-processed.
